(19) United States
(12) Patent Application Publication
(10) Pub. No.: US 2024/0285395 A1
(43) Pub. Date: Aug. 29, 2024

(54) PRESBYOPIA-CORRECTING OPHTHALMIC LENS WITH REDUCED VISUAL DISTURBANCES

(71) Applicant: Alcon Inc., Fribourg (CH)

(72) Inventors: Wangkuen LEE, Arlington, TX (US); Shinwook LEE, Arlington, TX (US); Sangyeol LEE, North Richland Hills, TX (US)

(21) Appl. No.: 18/581,631

(22) Filed: Feb. 20, 2024

Related U.S. Application Data
(60) Provisional application No. 63/486,567, filed on Feb. 23, 2023.

(51) Int. Cl. A61F 2/16

(52) U.S. Cl. CPC A61F 2/1618; A61F 2/1648; A61F 2002/1681

(57) ABSTRACT

An intraocular lens (IOL) includes a lens body having an anterior surface and a posterior surface disposed about an optical axis, and a progressive phase step structure formed on a refractive surface profile of at least one of the anterior surface or the posterior surface, the at least one of the anterior surface or the posterior surface having an outer zone, an inner zone, and a transition zone continuously connecting the outer zone and the inner zone. The refractive surface profile in the outer zone provides a base power, and the refractive surface profile in the inner zone provides an add power. The progressive phase step structure includes a first annular ridge structure within the inner zone, and a second annular ridge structure extending radially from the transition zone to the outer zone.

### CROSS-REFERENCE TO RELATED APPLICATIONS

This application claims priority to and benefit of U.S. Provisional Patent Application No. 63/486,567, filed Feb. 23, 2023, which is hereby assigned to the assignee hereof and hereby expressly incorporated by reference herein in its entirety as if fully set forth below and for all applicable purposes.

### BACKGROUND

The human eye in its simplest terms functions to provide vision by transmitting light through a clear outer portion called the cornea, and focusing the image by way of a lens onto a retina. The quality of the focused image depends on many factors including the size and shape of the eye, and the transparency of the cornea and lens. When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by a presbyopia-correcting intraocular lens (PC-IOL).

PC-IOLs are used for both refractive lens exchange and cataract surgery to replace the natural lens of the eye and correct refractive errors. Among them are extended depth of focus (EDOF) IOLs and diffractive multifocal IOLs. While the benefits of existing PC-IOLs are known, improvements to PC-IOL designs continue to improve outcomes and benefit patients.

### SUMMARY

Aspects of the present disclosure provide an ophthalmic lens, such as an intraocular lens (IOL) or a contact lens, including a lens body having an anterior surface and a posterior surface disposed about an optical axis, and a progressive phase step structure formed on a refractive surface profile of at least one of the anterior surface or the posterior surface, the at least one of the anterior surface or the posterior surface having an outer zone, an inner zone, and a transition zone continuously connecting the outer zone and the inner zone. The refractive surface profile in the outer zone provides a base power, the refractive surface profile in the inner zone provides an add power. The progressive phase step structure includes a first annular ridge structure within the inner zone, and a second annular ridge structure extending radially from the transition zone to the outer zone.

Aspects of the present disclosure also provide an ophthalmic lens, for example an intraocular lens (IOL), including a lens body having an anterior surface and a posterior surface disposed about an optical axis, and a progressive phase step structure formed on a refractive surface profile of at least one of the anterior surface or the posterior surface. The refractive surface profile and the progressive phase step structure are formed such as to provide continuous vision having a visual acuity of about 0.2 logMAR in a defocus range between 0 Diopter and −2.2 Diopter.

Aspects of the present disclosure further provide an intraocular lens (IOL) including a lens body having an anterior surface and a posterior surface disposed about an optical axis, and a progressive phase step structure formed on a refractive surface profile of at least one of the anterior surface or the posterior surface, the at least one of the anterior surface or the posterior surface having an outer zone, an inner zone, and a transition zone continuously connecting the outer zone and the inner zone. The refractive surface profile in the outer zone provides a base power, and the refractive surface profile in the inner zone provides an add power. The progressive phase step structure includes a first annular ridge structure within the inner zone, and a second annular ridge structure extending radially from the transition zone to the outer zone. The refractive surface profile and the progressive phase step structure are formed such as to provide continuous vision having a visual acuity of about 0.2 logMAR in a defocus range between 0 Diopter and −2.2 Diopter.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIG. 1A depicts a top view of an intraocular lens (IOL), according to certain embodiments.

FIG. 1B depicts a side view of a portion of the IOL of FIG. 1A, according to certain embodiments.

FIG. 2A depicts a refractive surface profile of an anterior surface of an IOL, according to certain embodiments.

FIG. 2B depicts a surface profile of a progressive phase step structure on the anterior surface of the IOL of FIG. 2A, according to certain embodiments.

FIG. 3A depicts a monocular visual acuity (VA) of an exemplary low visual disturbance (LVD) PC-IOL, according to certain embodiments.

FIGS. 3B, 3C, and 3D depict modulation transfer functions (MTFs) of an exemplary low visual disturbance (LVD) PC-IOL, according to certain embodiments.

FIG. 4 depicts an example system for designing, configuring, and/or forming an IOL, according to certain embodiments.

FIG. 5 depicts example operations for forming an IOL, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The embodiments described herein provide an ophthalmic lens, such as an intraocular lens (IOL), having a surface profile that produces a controlled variation of phase shifts in light waves passing through various regions of the IOL in a manner that extends the depth of focus, and methods and systems for fabricating the same. In certain embodiments, a lens surface of the IOL has a progressive phase step structure in conjunction with a refractive add power surface to produce continuous visual acuity from distance vision to near vision. The presbyopia-correcting intraocular lenses (PC-IOL) described herein may provide a full visual range performance (e.g., by maximizing the depth of focus to the near vision) without the use of a diffractive structure, while minimizing visual disturbances (VD), such as halos. Other example embodiments may include a contact lens having the described progressive phase step structure in conjunction with a refractive add power surface to provide continuous visual acuity from distance to near vision.

### Non-Diffractive Presbyopia-Correcting Intraocular Lenses (PC-IOL)

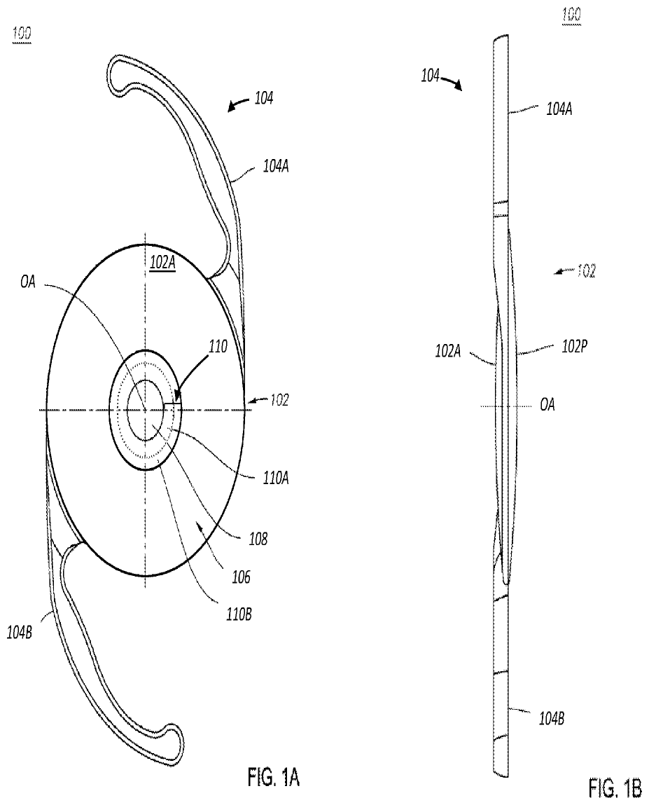

FIG. 1A depicts a top view of an intraocular lens (IOL) 100, according to certain embodiments. FIG. 1B depicts a side view of the IOL 100. The IOL 100 includes a lens body 102 and a haptic portion 104 that is coupled to a peripheral, non-optic portion of the lens body 102.

The lens body 102 has an anterior surface 102A and a posterior surface 102P that are disposed about an optical axis OA. The posterior surface 102P may have a smooth surface profile, for example, a smooth convex profile. On the anterior surface 102A, a progressive phase step structure is formed on a base surface profile of the anterior surface 102A. The anterior surface 102A includes an outer zone 106, an inner zone 108, and a transition zone 110 that continuously connects the outer zone 106 and the inner zone 108. The base surface in the outer zone 106 provides a base power appropriate for distance vision correction (and considered as a zero add power). The base surface in the inner zone 108 provides an add power appropriate for near vision correction. The transition zone 110 may have two or more sub-zones 110A and 110B. A progressive phase step structure may be formed on the anterior surface 102A in one or more of the sub-zones 110A and 110B of the transition zone 110. The progressive phase step structure produces varying phase shifts of light waves passing through various regions or zones of the lens body 102. Constructive interference between the light waves having varying amounts of phase shifts produces an extended depth-of-focus. An overall surface profile Z_{A}(r) (described as a sag of a point on the anterior surface 102A at a radial distance r from a point on the anterior surface 102A at the optical axis OA) of the anterior surface 102A is thus a sum of a refractive surface profile Z_{RP}(r) and a surface profile Z_{PS}(r) of the progressive phase step structure, Z_{A}(r)=Z_{RP}(r)+Z_{PS}(r), as described in detail below.

Although the refractive surface profile and the progressive phase step structure are formed only on the anterior surface 102A of the lens body 102 in the example described herein, the refractive surface profile and the progressive phase step structure may be formed on a posterior surface 102P of the lens body 102, or on both of the anterior surface 102A and the posterior surface 102P of the lens body 102.

It is noted that the shape and curvatures of the lens body 102 are shown for illustrative purposes only and that other shapes and curvatures are also within the scope of this disclosure. For example, the lens body 102 shown in FIG. 1B has a bi-convex shape. In other examples, the lens body 102 may have a plano-convex shape, a convexo-concave shape, or a plano-concave shape.

The lens body 102 may be fabricated of biocompatible material, such as modified poly (methyl methacrylate) (PMMA), modified PMMA hydrogels, hydroxy-ethyl methacrylate (HEMA), PVA hydrogels, other silicone polymeric materials, and hydrophobic acrylic polymeric materials, for example, AcrySof® and Clareon® materials, available from Alcon, Inc., Fort Worth, Texas. The lens body 102 has a diameter of between about 4.5 mm and about 7.5 mm, for example, about 6.0 mm.

The haptic portion 104 includes radially-extending struts (also referred to as “haptics”) 104A and 104B that are coupled (e.g., glued or welded) to the peripheral portion of the lens body 102 or molded along with a portion of the lens body 102, and thus extend radially from the lens body 102 to engage the perimeter wall of the capsular sac of the eye to maintain the lens body 102 in a desired position in the eye. The haptics 104A and 104B may be fabricated of biocompatible material, such as modified poly (methyl methacrylate) (PMMA), modified PMMA hydrogels, hydroxy-ethyl methacrylate (HEMA), PVA hydrogels, other silicone polymeric materials, and hydrophobic acrylic polymeric materials, for example, AcrySof® and Clareon® materials, available from Alcon, Inc., Fort Worth, Texas. The haptics 104A and 104B typically have radial-outward ends that define arcuate terminal portions. The terminal portions of the haptics 104A and 104B may be separated by a length of between about 6 mm and about 22 mm, for example, about 13 mm. The haptics 104A and 104B have a particular length so that the terminal portions create a slight engagement pressure when in contact with the equatorial region of the capsular sac after being implanted. While FIG. 1A depicts one example configuration of the haptics 104A and 104B, any plate haptics or other types of haptics can be used.

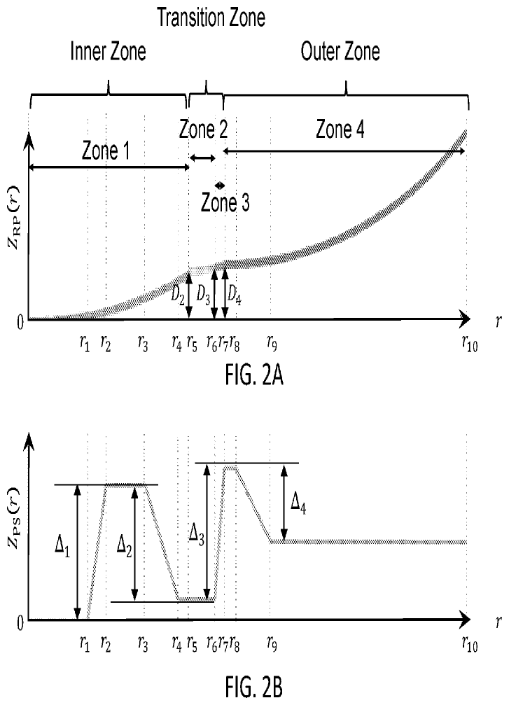

FIG. 2A depicts a refractive surface profile Z_{RP}(r) of the anterior surface 102A. The refractive surface profile Z_{RP}(r) includes a refractive surface profile Z_{Zone 1}(r) of the inner zone 108 (also referred to as “Zone 1,” 0≤r<r₅), which provides an add power for near vision correction, and a refractive surface profile Z_{Zone 4}(r) of the outer zone 106 (also referred to as “Zone 4,” r₇≥r<r₁₀), which provides a base power for distance vision correction. The inner zone 108, with the refractive surface profile Z_{Zone 1}(r), and the outer zone 106, with the refractive surface profile Z_{Zone 4}(r), are continuously connected via the transition zone 110 (r₅≤r<r₇) with the refractive surface profiles Z_{Zone 2}(r) and Z_{Zone 3}(r). The transition zone 110 of the anterior surface 102A may include two or more zones, including a sub-zone 110A (also referred to as “Zone 2,” r₅≤r<r₆) and a sub-zone 110B (also referred to as “Zone 3,” r₆≤r<r₇) encircling the sub-zone 110A. Adding sag offsets D₂, D₃, and D₄ to the refractive surface profiles Z_{Zone 1}(r), Z_{Zone 2}(r), Z_{Zone 3}(r), and Z_{Zone 4} (r) produces the refractive surface profile Z_{RP}(r) such that there are no discontinuities at the boundaries between the adjacent zones. For example, the refractive surface profile Z_{RP}(r) may be defined as:

$${Z}_{R P} ( r ) = \{ \begin{matrix}{Z}_{Zone 1} ( r ) , & ( 0 \leq r < {r}_{5} ) \\ {D}_{2} + {Z}_{Zone 2} ( r ) , & ( {r}_{5} \leq r < {r}_{6} ) \\ {D}_{3} + {Z}_{Zone 3} ( r ) , & ( {r}_{6} \leq r < {r}_{7} ) \\ {D}_{4} + {Z}_{Zone 4} ( r ) , & ( {r}_{7} \leq r < {r}_{10} )\end{matrix} ,$$

where the refractive surface profiles Z_{Zone 1}(r), Z_{Zone 2}(r), Z_{Zone 3}(r), and Z_{Zone 4} (r) are defined as:

$${Z}_{Zone 1} ( r ) = \frac{{c}_{1} {r}^{2}}{1 + \sqrt{1 - ( 1 + {k}_{1} ) {c}_{1}^{2} {r}^{2}}} , \\ {Z}_{Zone 2} ( r ) = \frac{{c}_{2} {r}^{2}}{1 + \sqrt{1 - ( 1 + {k}_{2} ) {c}_{2}^{2} {r}^{2}}} , \\ {Z}_{Zone 3} ( r ) = \frac{{c}_{3} {r}^{2}}{1 + \sqrt{1 - ( 1 + {k}_{3} ) {c}_{3}^{2} {r}^{2}}} , and \\ {Z}_{Zone 4} ( r ) = \frac{{c}_{4} {r}^{2}}{1 + \sqrt{1 - ( 1 + {k}_{4} ) {c}_{4}^{2} {r}^{2}}} + {A}_{4} {r}^{4} + {A}_{6} {r}^{6} .$$

Curvature c₁ and conic constant k₁ are determined based on the add power desired for the inner zone 108. The coefficients c₄ and k₄ are determined based on the base power for the outer zone 106. The transition zone parameters c₂, k₂, c₃, and k₃ are determined by optimizing the design for better visual acuity (VA) performance. Smooth and continuous VA performance between intermediate vision around-1.0 Diopter and near vision around-2.0 Diopter can be provided by the embodiments herein by optimizing those refractive zone parameters in conjunction with the progressive phase structure. Coefficients A₄ and A₆ are the fourth and sixth order aspheric coefficients.

The outer radius r₅ of the inner zone 108 may be between about 0.95 mm and about 1.5 mm, for example, about 1.1 mm. The outer radius r₆ of the sub-zone 110A of the transition zone 110 may be between about 1.0 mm and about 1.5 mm, for example, about 1.25 mm. The outer radius r₇ of the sub-zone 110B of the transition zone 110 may be between about 1.25 mm and about 2.05 mm, for example, about 1.3 mm. A radius of curvature (1/c₄) of the base profile Z_{Base}(r)=Z_{Zone4}(r) (also referred to as a “base radius”) may be between about 5.5 mm and about 95 mm. A radius of curvature (1/c₁) of the refractive surface profile Z_{Zone 1}(r) and a radius of curvature (1/c₃) of the refractive surface profile Z_{Zone 3}(r) may each be between about the base radius minus 10 mm and about the base radius. A radius of curvature (1/c₂) of the refractive surface profile Z_{Zone 2}(r) may be between about the base radius and about the base radius plus 10 mm, which is greater than the radius of curvature (1/c₃) of the refractive surface profile Z_{Zone 3}(r). The conic constants k₁, k₂, k₃, and k₄ may be between −100 and +100, between −50 and +50, between −50 and +5−, and −2500 and +2500, respectively. The coefficient A₄ may be between-5.0×10⁻⁴ mm⁻³ and +5.0×10⁻⁴ mm⁻³. The coefficient A₆ may be between −5.0×10⁻⁴ mm⁻⁵ and +5.0×10⁻⁴ mm⁻⁵.

FIG. 2B depicts a surface profile Z_{PS}(r) of the progressive phase step structure on the anterior surface 102A. The surface profile Z_{PS}(r) includes progressive steps described as:

$${Z}_{P S} ( r ) = \{ \begin{matrix}0 , & ( 0 \leq r < {r}_{1} ) \\ \frac{{Δ}_{1}}{( {r}_{2} - {r}_{1} )} ( r - {r}_{1} ) , & ( {r}_{1} \leq r < {r}_{2} ) \\ {Δ}_{1} , & ( {r}_{2} \leq r < {r}_{3} ) \\ {Δ}_{1} - \frac{{Δ}_{2}}{( {r}_{4} - {r}_{3} )} ( r - {r}_{3} ) , & ( {r}_{3} \leq r < {r}_{4} ) \\ {Δ}_{1} - {Δ}_{2} , & ( {r}_{4} \leq r < {r}_{6} ) \\ {Δ}_{1} - {Δ}_{2} + \frac{{Δ}_{3}}{( {r}_{7} - {r}_{6} )} ( r - {r}_{6} ) , & ( {r}_{6} \leq r < {r}_{7} ) \\ {Δ}_{1} - {Δ}_{2} + {Δ}_{3} , & ( {r}_{7} \leq r \leq {r}_{8} ) \\ {Δ}_{1} - {Δ}_{2} + {Δ}_{3} - \frac{{Δ}_{4}}{( {r}_{9} - {r}_{8} )} ( r - {r}_{8} ) & ( {r}_{8} \leq r \leq {r}_{9} ) \\ {Δ}_{1} - {Δ}_{2} + {Δ}_{3} - {Δ}_{4} , & ( {r}_{9} \leq r \leq {r}_{10} )\end{matrix} ,$$

where Δ₁ is a step height of a zone r₂≤r<r₃ within the inner zone 108 (Zone 1) relative to the optical axis OA (r=0), Δ₂ is a step height of a zone r₄≤r<r₆ that spans over the inner zone 108 (Zone 1) and the sub-zone 110A (Zone 2) relative to the zone r₂≤r<r₃, Δ₃ is a step height of a zone r₇≤r≤r₈ within the outer zone 106 (Zone 4) relative to the zone r₄≤r<r₆, and Δ₄ is a step height of a zone r₉≤r<r₁₀ within the outer zone 106 (Zone 4) relative to the zone r₇≤r≤r₈.

As shown in FIG. 2B, the progressive phase step structure includes two annular ridge structures, a first annular ridge structure extending radially from r=r₁ (within the inner zone 108) to r=r₄ (within the inner zone 108), and a second annular ridge structure extending radially from r=r₆ (at the boundary between the sub-zone 110A and 110B within the transition zone 110) to r=r₉ (within the outer zone 106). The first annular ridge structure increases in height radially from r=r₁ to r=r₂, and decreases in height radially from r=r₃ to r=r₄, which is less than r=r₅ (at the boundary between the inner zone 108 and the transition zone 110). The second annular ridge structure increases in height radially from r=r₆ (at the boundary between the sub-zone 110A and the sub-zone 110B) to r=r₇ (at the boundary between the transition zone 110 and the outer zone 106), and decreases in height radially from r=r₈ to r=r₉.

Moving radially outward from the optical axis OA may result in four phase shift steps. Constructive interference between the light waves having varying amounts of phase shifts produce an extended depth-of-focus.

Ranges of the parameters Δ₁, Δ₂, Δ₃, Δ₄, r₁, r₂, r₃, r₄, r₈, r₉, and r₁₀. are shown below.

| Parameters | Range | Unit |
|---|---|---|
| Δ₁ | ±5 | μm |
| Δ₂ | ±2 | μm |
| Δ₃ | ±10 | μm |
| Δ₄ | ±5 | μm |
| r₁ | 0.3-1.5 | mm |
| r₂ | 0.4-1.5 | mm |
| r₃ | 0.6-1.5 | mm |
| r₄ | 0.8-1.5 | mm |
| r₈ | 1.3-2.1 | mm |
| r₉ | 1.4-2.3 | mm |
| r₁₀ | 3 | mm |

### Examples

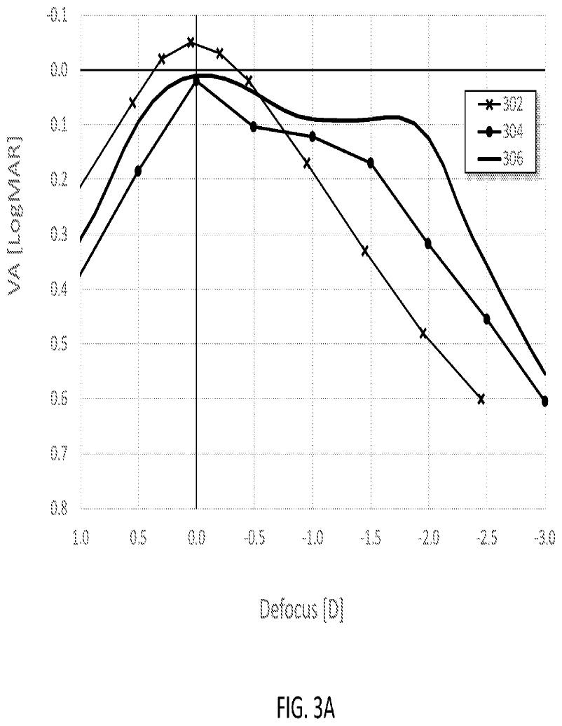

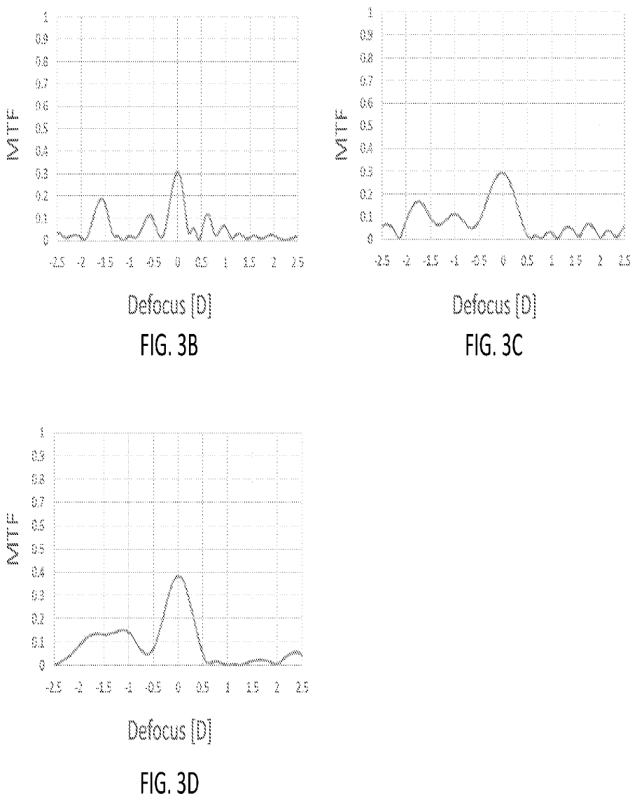

FIG. 3A depicts a monocular visual acuity (VA) of an exemplary low visual disturbance (LVD) PC-IOL with the surface profile Z_{RP}(r)+Z_{PS}(r) shown in FIGS. 2A and 2B measured in LogMAR (logarithm of the minimum angle of resolution) scores. A 0 logMAR score corresponds to a score of 20/20 on a Snellen chart, or 100 lp/mm (line pairs per millimeter) spatial resolution (also referred to as “spatial frequency”). A 0.4 logMAR score corresponds to a score of 20/50 on a Snellen chart, or 40 lp/mm. The monocular VA was evaluated using a 3-mm (photopic) aperture to determine a depth of focus (also referred to as “defocus”) for the lens.

In FIG. 3A, a monocular VA 302 of a typical monofocal IOL (creating a single focal point) and a monocular VA 304 of a typical EDOF IOL (creating a single-elongated focal point) are shown together with a simulated result 306 of a monocular VA of an exemplary LVD PC-IOL. The monocular VA 304 of the typical EDOF IOL shows an extended depth of focus as compared to the monocular VA 302 of the typical monofocal IOL. The monocular VA 306 of the exemplary LVD PC-IOL shows a similar extended depth of focus to the monocular VA 304 of the typical EDOF IOL, but significant enhancement of the VA at an intermediate distance (about-1.5 Diopter) and a near distance (about-2 Diopter), as compared to the typical EDOF IOL. As can be seen in FIG. 3A, the exemplary LVD PC-IOL shows continuous ranges of vision from distance vision to near vision (i.e., VA above, or better than, 0.1 logMAR from distance vision to a near vision up to −1.8 Diopter, and VA above 0.2 logMAR from distance vision 0 Diopter to a near vision up to −2.2 Diopter).

FIGS. 3B, 3C, and 3D depict modulation transfer functions (MTFs) of an exemplary low visual disturbance (LVD) PC-IOL with the surface profile Z_{RP}(r)+Z_{PS}(r) shown in FIGS. 2A and 2B, evaluated at a focus plane at 100 lp/mm (corresponding to VA of 20/20), 67 lp/mm (corresponding to VA of 20/30), and at 50 lp/mm (corresponding to VA of 20/40), respectively.

### System for Designing an IOL

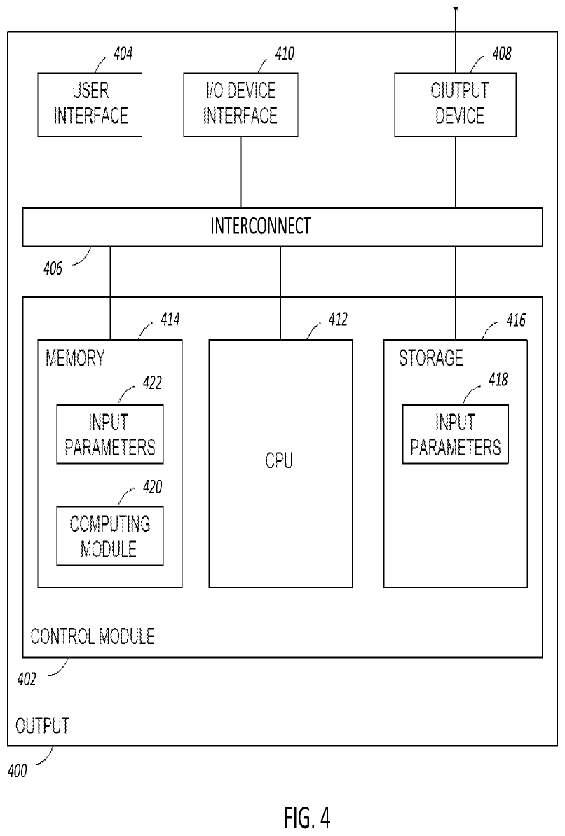

FIG. 4 depicts an exemplary system 400 for designing, configuring, and/or forming an IOL, such as LVD PC-IOLs described herein. As shown, the system 400 includes, without limitation, a control module 402, a user interface display 404, an interconnect 406, an output device 408, and at least one I/O device interface 410, which may allow for the connection of various I/O devices (e.g., keyboards, displays, mouse devices, pen input, etc.) to the system 400.

The control module 402 includes a central processing unit (CPU) 412, a memory 414, and a storage 416. The CPU 412 may retrieve and execute programming instructions stored in the memory 414. Similarly, the CPU 412 may retrieve and store application data residing in the memory 414. The interconnect 406 transmits programming instructions and application data, among CPU 412, the I/O device interface 410, the user interface display 404, the memory 414, the storage 416, output device 408, etc. The CPU 412 can represent a single CPU, multiple CPUs, a single CPU having multiple processing cores, and the like. Additionally, in certain embodiments, the memory 414 represents volatile memory, such as random-access memory. Furthermore, in certain embodiments, the storage 416 may be non-volatile memory, such as a disk drive, solid state drive, or a collection of storage devices distributed across multiple storage systems.

As shown, the storage 416 includes input parameters 418, including any of the parameters used as input in the equations provided herein (e.g., equations described in relation to FIGS. 2A and 2B). The input parameters 418 include a lens base power and an add power. The memory 414 includes a computing module 420 for computing control parameters, such as outer radii of various zones and step heights of a surface profile of a surface (e.g., anterior surface) of a lens. In addition, the memory 414 includes input parameters 422.

In certain embodiments, input parameters 422 correspond to input parameters 418 or at least a subset thereof. In certain embodiments, during the computation of the control parameters, the input parameters 422 are retrieved from the storage 416 and executed in the memory 414. In such an example, the computing module 420 comprises executable instructions for computing the control parameters, based on the input parameters 422. In certain other embodiments, input parameters 422 correspond to parameters received from a user through user interface display 404. In such embodiments, the computing module 420 comprises executable instructions for computing the control parameters, based on information received from the user interface display 404.

In certain embodiments, the computed control parameters, are output via the output device 408 to a lens manufacturing system that is configured to receive the control parameters and form a lens accordingly. In certain other embodiments, the system 400 itself is representative of at least a part of a lens manufacturing systems. In such embodiments, the control module 402 then causes hardware components (not shown) of system 400 to form the lens according to the control parameters. The details of a lens manufacturing system are known to one of ordinary skill in the art and are omitted here for brevity.

### Method for Forming an IOL

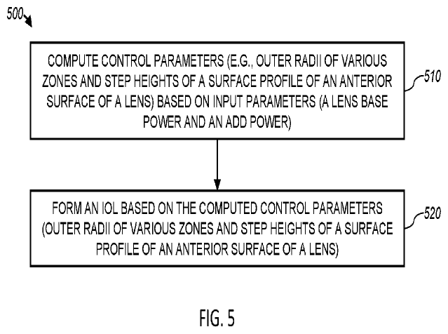

FIG. 5 depicts example operations 500 for forming an IOL (e.g., IOL 100). In some embodiments, the step 510 of operations 500 is performed by one system (e.g., the system 400) while step 520 is performed by a lens manufacturing system. In some other embodiments, both steps 510 and 520 are performed by a lens manufacturing system.

At step 510, control parameters (e.g., outer radii of various zones and step heights of a surface profile of a surface (e.g., anterior surface) of a lens) are computed based on input parameters (e.g., a lens base power and an add power). The computations performed at step 510 are based on one or more of the embodiments described herein. A variety of optimization techniques or algorithms may be used for selecting an appropriate outer radii for various zones and step heights of a surface profile of a surface (e.g., anterior surface) of a lens. For example, a method may be used to numerically minimize an error function for calculating the difference between the target and achieved visual acuity, by varying design parameters.

At step 520, an IOL (e.g., IOL 100) is formed based on the computed control parameters (e.g., outer radii of various zones and step heights of a surface profile of an anterior surface of a lens), using appropriate methods, systems, and devices typically used for manufacturing lenses, as known to one of ordinary skill in the art.

The embodiments described herein provide presbyopia-correcting IOLs in which continuous vision from distance to near is achieved, while simultaneously avoiding the introduction of or at least reducing visual disturbances (e.g., halos, glare) more commonly associated with diffractive presbyopia-correcting IOLs. By providing continuous distance-to-near vision, the exemplary embodiments of the low visual disturbance PC-IOLs may in some instances provide a greater depth of focus than some other EDOF IOLs. Avoiding the visual disturbances (VDs), such as halo or glare, may also avoid reductions in visual acuity and contrast sensitivity.

While the foregoing is directed to embodiments of the present disclosure, other and further embodiments of the disclosure may be devised without departing from the basic scope thereof, and the scope thereof is determined by the claims that follow.

1. An ophthalmic lens, comprising:
a lens body having an anterior surface and a posterior surface disposed about an optical axis; and
a progressive phase step structure formed on a refractive surface profile of at least one of the anterior surface or the posterior surface, the at least one of the anterior surface or the posterior surface having an outer zone, an inner zone, and a transition zone continuously connecting the outer zone and the inner zone, wherein:
the refractive surface profile in the outer zone provides a base power;
the refractive surface profile in the inner zone provides an add power in the inner zone; and
the progressive phase step structure comprises:
a first annular ridge structure within the inner zone; and
a second annular ridge structure extending radially from the transition zone to the outer zone.

2. The ophthalmic lens of claim 1, wherein the refractive surface profile and the progressive phase step structure are formed such as to provide continuous vision having a visual acuity of about 0.2 logMAR in a defocus of between 0 Diopter and −2.2 Diopter.

3. The ophthalmic lens of claim 1, wherein:
the transition zone comprises a first sub-zone and a second sub-zone encircling the first sub-zone; and
a radius of curvature of the refractive surface profile in the first sub-zone is greater than a radius of curvature of the refractive surface profile in the second sub-zone.

4. The ophthalmic lens of claim 3, wherein:
the first annular ridge structure increases in height radially from a first radial distance from the optical axis to a second radial distance from the optical axis, and decreases in height radially from a third radial distance from the optical axis to a fourth radial distance from the optical axis; and
the fourth radial distance is less than a fifth radial distance from the optical axis at a boundary between the inner zone and the transition zone.

5. The ophthalmic lens of claim 4, wherein:
the second annular ridge structure increases in height radially from a sixth radial distance from the optical axis to a seventh radial distance from the optical axis, and decreases in height radially from an eighth radial distance from the optical axis to a ninth radial distance from the optical axis,
the sixth radial distance is at a boundary between the first sub-zone and the second sub-zone;
the seventh radial distance is at a boundary between the transition zone and the outer zone.

6. The ophthalmic lens of claim 1, wherein the lens body comprises a hydrophobic acrylic polymeric material.

7. The ophthalmic lens of claim 1, further comprising one or more haptics coupled to the lens body.

8. An ophthalmic lens, comprising:
a lens body having an anterior surface and a posterior surface disposed about an optical axis; and
a progressive phase step structure formed on a refractive surface profile of at least one of the anterior surface or the posterior surface, wherein:
the refractive surface profile and the progressive phase step structure are formed such as to provide continuous vision having a visual acuity of better than 0.2 logMAR in a defocus range between 0 Diopter and −2.2 Diopter.

9. The ophthalmic lens of claim 8, wherein:
the at least one of the anterior surface or the posterior surface comprises:
an outer zone, in which the refractive surface profile provides a base power;
an inner zone, in which the refractive surface profile provides an add power; and
a transition zone that continuously connects the outer zone and the inner zone; and
the progressive phase step structure comprises:
a first annular ridge structure within the inner zone; and
a second annular ridge structure extending radially from the transition zone to the outer zone.

10. The ophthalmic lens of claim 9, wherein:
the transition zone comprises a first sub-zone and a second sub-zone encircling the first sub-zone, and
a radius of curvature of the refractive surface profile in the first sub-zone is greater than a radius of curvature of the refractive surface profile in the second sub-zone.

11. The ophthalmic lens of claim 10, wherein:
the first annular ridge structure increases in height radially from a first radial distance from the optical axis to a second radial distance from the optical axis, and decreases in height radially from a third radial distance from the optical axis to a fourth radial distance from the optical axis; and
the fourth radial distance is less than a fifth radial distance from the optical axis at a boundary between the inner zone and the transition zone.

12. The ophthalmic lens of claim 11, wherein:
the second annular ridge structure increases in height radially from a sixth radial distance from the optical axis to a seventh radial distance from the optical axis, and decreases in height radially from an eighth radial distance from the optical axis to a ninth radial distance from the optical axis;
the sixth radial distance is at a boundary between the first sub-zone and the second sub-zone;
the seventh radial distance is at a boundary between the transition zone and the outer zone.

13. The ophthalmic lens of claim 9, wherein the lens body comprises hydrophobic acrylic polymeric material.

14. The ophthalmic lens of claim 9, further comprising one or more haptics coupled to the lens body.

15. An intraocular lens (IOL), comprising:
a lens body having an anterior surface and a posterior surface disposed about an optical axis; and
a progressive phase step structure formed on a refractive surface profile of at least one of the anterior surface or the posterior surface, the at least one of the anterior surface or the posterior surface having an outer zone, an inner zone, and a transition zone continuously connecting the outer zone and the inner zone, wherein
the refractive surface profile in the outer zone provides a base power;
the refractive surface profile in the inner zone provides an add power, and
the progressive phase step structure comprises:
a first annular ridge structure within the inner zone; and
a second annular ridge structure extending radially from the transition zone to the outer zone,
wherein the refractive surface profile and the progressive phase step structure are formed such as to provide continuous vision having a visual acuity of better than 0.2 logMAR in a defocus range between 0 Diopter and −2.2 Diopter.

16. The IOL of claim 15, wherein:
the transition zone comprises a first sub-zone and a second sub-zone encircling the first sub-zone, and
a radius of curvature of the refractive surface profile in the first sub-zone is greater than a radius of curvature of the refractive surface profile in the second sub-zone.

17. The IOL of claim 16, wherein:
the first annular ridge structure increases in height radially from a first radial distance from the optical axis to a second radial distance from the optical axis, and decreases in height radially from a third radial distance from the optical axis to a fourth radial distance from the optical axis; and
the fourth radial distance is less than a fifth radial distance from the optical axis at a boundary between the inner zone and the transition zone.

18. The IOL of claim 17, wherein:
the second annular ridge structure increases in height radially from a sixth radial distance from the optical axis to a seventh radial distance from the optical axis, and decreases in height radially from an eighth radial distance from the optical axis to a ninth radial distance from the optical axis;
the sixth radial distance is at a boundary between the first sub-zone and the second sub-zone;
the seventh radial distance is at a boundary between the transition zone and the outer zone.

19. The IOL of claim 15, wherein the lens body comprises hydrophobic acrylic polymeric material.

20. The IOL of claim 15, further comprising one or more haptics coupled to the lens body.
